(19) United States
(12) Patent Application Publication
(10) Pub. No.: US 2008/0167575 A1
(43) Pub. Date: Jul. 10, 2008

(54) Audiologist Equipment Interface User Database For Providing Aural Rehabilitation Of Hearing Loss Across Multiple Dimensions Of Hearing

(75) Inventors: John Cronin, Jericho, VT (US); Tushar Narsana, Naperville, IL (US); Cindy Timblin, Bristol, VT (US)

Correspondence Address: MCCARTER & ENGLISH, LLP, FOUR GATEWAY CENTER, 100 MULBERRY STREET, NEWARK, NJ, 07102, US

(73) Assignee: JOHNSON & JOHNSON CONSUMER COMPANIES, INC., Skillman, NJ (US)

(21) Appl. No.: 11/570,455

(22) PCT Filed: Jun. 13, 2005
(86) PCT No.: PCT/US2005/020827

§ 371 Date: Oct. 3, 2007

Related U.S. Application Data
(60) Provisional application No. 60/579,486, filed on Jun. 14, 2004.

(51) Int. Cl. A61B 5/12; G06F 19/00

(52) U.S. Cl. 600/559

(57) ABSTRACT

An audiometer system (100) includes a user (105), a sound room (110), a Speaker (115), a pair of headphones (120), a pair of leads (125) and (130), a button (135), and an audiometer (140). User (105) is an individual on whom a hearing test is to be administered. User (105) is an individual on whom a hearing test is to be administrated. User (105) wears headphones (120) in sound room (100). An audiologist conducts a Hearing test by operating audiometer (150). Audiometer (140) produces a hearing test by operating audiometer (140). Audiometer (140) produces the required tones at the desires frequency and amplitudes, according to adjustments made to frequency adjust (150) and amplitude adjusts (150). Frequency adjust (145) and amplitude adjust (150) can be rotary or push button adjustments.

### CROSS REFERENCE TO RELATED APPLICATION

This application claims the benefit of U.S. Provisional Application No. 60/579,486 filed Jun. 14, 2004, assigned to the assignee of this application and incorporated by reference herein.

### FIELD OF THE INVENTION

The present invention relates to hearing testing interface systems. More particularly, the present invention relates to an interface between standard, older audiologist's equipment and a modern personal computer system for advanced data analysis and advanced audiological testing and simulation.

### BACKGROUND OF THE INVENTION

More than twenty-five million Americans have hearing loss, including one out of four people older than sixty-five. Hearing loss may come from infections, strokes, head injuries, some medicines, tumors, other medical problems, or even excessive earwax. It can also result from repeated exposure to very loud noise, such as music, power tools, or jet engines. Changes in the way the ear works as a person ages can also affect hearing.

In a well-known method of testing hearing loss in individuals, the threshold of the individual's hearing is typically measured using a calibrated sound-stimulus-producing device and calibrated headphones. The measurement of the threshold of hearing takes place in an isolated sound room, usually a room where there is very little audible ambient noise. The sound-stimulus-producing device and the calibrated headphones used in the testing are known as an audiometer.

There are several limitations associated with conventional audiometers typically used to conduct a hearing test. One limitation is that the audiologist must manually record the frequency and amplitude of each tone produced and the patient's responses to the various tones. This manual recording can be tedious and is often prone to error. A solution to this problem is to use a computer system to record the hearing test data. Computer systems are routinely used in health care settings for data tracking, data analysis, and record keeping. However, older, conventional audiometers do not typically interface with existing computer systems. To take advantage of the data management capabilities of a computer system, the audiologist must manually re-enter the data into the computer. Thus, there exists a need for a means to automatically extract hearing test data from a conventional audiometer without manually re-entering the data.

Another limitation of conventional audiometers is that they are typically used to conduct simple frequency versus amplitude tests and do not take into account other hearing issues such as speech intelligibility issues (i.e., understanding spoken words and sentences). For example, even though an individual may have some hearing loss, he or she may be able to function quite normally, whereas others may have limitations in understanding certain spoken words. Playing pre-recorded words and sentences instead of tones can test speech intelligibility. However, older, conventional audiometers are typically limited to producing tones of varying frequency and amplitude and lack the ability to play pre-recorded words or sentences. A variety of audiological programs, sound “.wav” files, and speech and other sound simulations files are available to audiologists from central hearing health databases. Thus, there exists a need to provide a means to access available hearing testing programs and extend the hearing testing capabilities of older, conventional audiometers.

Another limitation of older, conventional audiometers is that they require the audiologist to manually adjust the frequency and amplitude to produce a range of tones suitable for conducting a hearing test. Typically, audiometers produce tones at frequencies between 125 Hz and 20 kHz and at amplitudes between −10 dB and 110 dB. This wide range of frequencies and amplitudes requires frequent adjustments be made by the audiologist to effectively conduct a hearing test. Thus, there exists a need for a means to automate an older, conventional audiometer to produce a range of tones of varying frequencies and amplitudes.

A wide variety of audiometers exist and are implemented by audiologists to test a patient's hearing health; some of these audiometers date back several decades and are not capable of interfacing with existing computer systems and computer networks. It is desirable to enable audiologists to practice the present invention with minimal investment and upgrades to existing equipment.

### SUMMARY OF THE INVENTION

It is therefore an object of the present invention to be able to extract audiological measurements from an older audiometer automatically without manually reentering data.

It is another object of the present invention to be able to drive the older audiometer with programs from a modern computing device.

It is yet another object of the present invention to provide an interface that enables conventional, non-PC compatible audiometers to be used during the practice of the present invention.

It is yet another object of the present invention to be able to automate an older audiometer with modern computing devices.

The present invention relates to a computer-interfaced audiometer system and a method of using the system. More particularly, the present invention relates to a computer interface system that is used to connect a conventional audiometer to a computer system. The computer-interfaced audiometer system uses computer directed programs to automatically record hearing test data, to provide extended hearing testing capabilities, and to interface with other computer systems and central databases so as to ensure rapid and accurate hearing health assessments. The computer-interfaced audiometer system also provides automated operation for conducting a hearing test.

Thus, the present invention provides for an interface for an audiometer, wherein the audiometer generates analog right and left tone signals at respective right and left signal outputs, the interface comprising:

an interface for coupling the right and left signal outputs, wherein the interface includes an analog to digital converter (“ADC”) for converting analog tone signals to digital tone data;

a controller including a processor and a memory, wherein the controller is coupled to the interface, a digital signal processor (“DSP”), a sound card, a tone output and an operator input;

wherein the processor is selectively controllable to operate the interface in a legacy mode and a processor control mode,

wherein in the legacy mode the processor routes the analog tone signals received at the interface to the tone output, and

wherein in the processor control mode the processor transmits the digital tone data to the DSP, wherein the DSP based on the digital tone data generates frequency and amplitude data corresponding to the analog tone signals represented by the digital tone data, stores the frequency and amplitude data in the memory and transmits the digital tone data to the sound card.

In a preferred embodiment of the interface, the controller is coupled to an input control port of the audiometer and the processor is selectively controllable to operate the interface in an automated processor control mode, wherein in the automated processor control mode the processor and the DSP operate as in the processor control mode and the processor further uses control data transmitted from the operator input to control (e.g., the frequency and amplitude of) generation of analog tone signals at the audiometer.

In a further preferred embodiment of the interface, the DSP in the processor control mode modifies the digital tone data with respect to at least one of amplitude and frequency characteristics of a corresponding analog tone signal.

In a still further preferred embodiment of the interface, the interface includes a network communications interface and the DSP generates sound data signals for transmission to the sound card based on hearing testing data received at the network interface.

In a further preferred embodiment of the interface, a user input is coupled to the controller and the processor stores in the memory data transmitted from the user input.

### BRIEF DESCRIPTION OF THE DRAWINGS

Other objects and advantages of the present invention will be apparent from the following detailed description of the presently preferred embodiments, which description should be considered in conjunction with the accompanying drawings in which like references indicate similar elements and in which:

FIG. 1 is a block diagram illustrating a conventional audiometer system.

FIG. 2 is a block diagram illustrating a computer-interfaced audiometer system according to the present invention.

FIG. 3 is a circuit diagram illustrating an equipment interface device.

FIG. 4 is a block diagram of an audiometer card for use in a computer-interfaced audiometer system.

FIG. 5 is a table illustrating an individual's hearing profile at specific amplitudes for numerous frequencies.

FIG. 6 is an illustrative example of a computer-interfaced audiometer graphical user interface.

FIG. 7 is a flow chart illustrating a method of running a standard hearing test using a computer-interfaced audiometer system.

### DESCRIPTION OF THE INVENTION

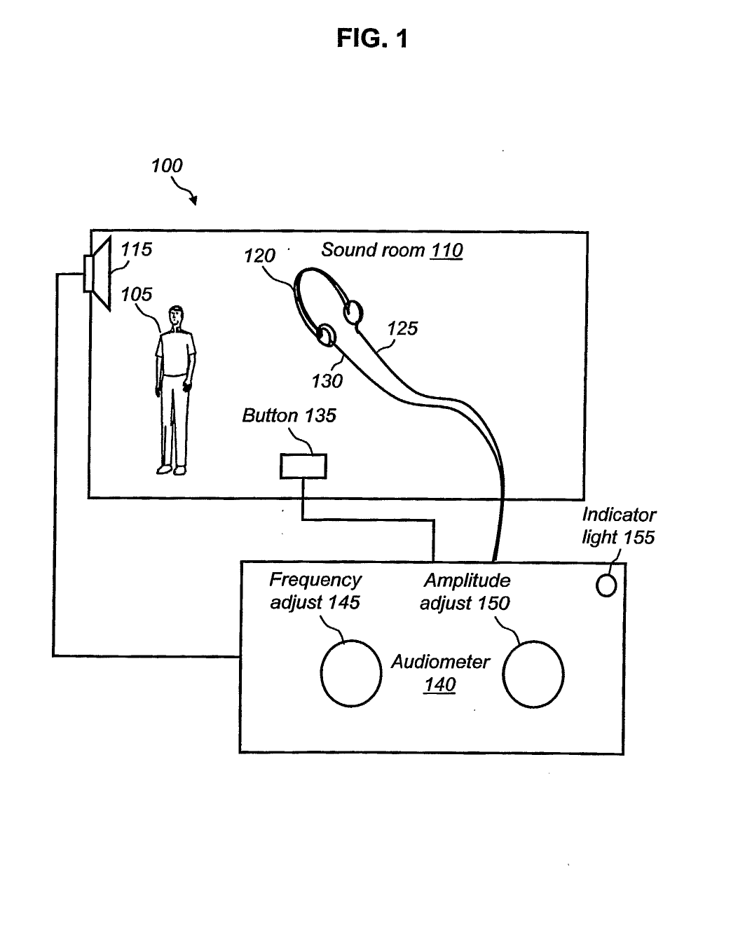

A variety of audiometers and their typical use have been described. FIG. 1 illustrates an example of a conventional audiometer system 100. System 100 includes a user 105, a sound room 110, a speaker 115, a pair of headphones 120, a pair of leads 125 and 130, a button 135, and an audiometer 140.

User 105 is an individual on whom a hearing test is to be administered. User 105 is a generally any individual, but more specifically, an individual in the more than 10% of the population (e.g., twenty-five million Americans) that have hearing loss, including one out of four people older than sixty-five.

Sound room 110 is any soundproof room that provides a suitable environment for a hearing test.

Speaker 115 and headphones 120 provide a means for administering a range of tones for testing the hearing of user 105. In a preferred example, user 105 is wearing headphones 120. Leads 125 and 130 connect headphones 120 to audiometer 140. Leads 125 and 130 provide a means to selectively test the right or left ear of user 105, respectively.

Button 135 is a response button that is pressed by user 105 to indicate that a sound has been heard.

Audiometer 140 is typically a conventional device certified by the American National Standards Institute (ANSI) for use by an audiologist to test an individual's hearing (i.e., user 105). Audiometer 140 further includes a frequency adjust 145, an amplitude adjust 150, and an indicator light 155. Audiometer 140 generates pure tones used in conducting a hearing test.

In operation, user 105 wears headphones 120 in sound room 110. An audiologist conducts a hearing test by operating audiometer 140. Audiometer 140 produces the required tones at the desired frequencies and amplitudes, according to adjustments made to frequency adjust 145 and amplitude adjust 150. Frequency adjust 145 and amplitude adjust 150 can be rotary or push-button adjustments. Typically, audiometer 140 produces tones at frequencies between 125 Hz and 20 kHz and at amplitudes between −10 dB and 110 dB. The audiologist activates audiometer 140 to deliver test tones to user 105 via headphones 120. Upon hearing a tone, user 105 presses button 135 and a positive signal is send to audiometer 140. The positive signal is observed by the audiologist as an illumination of indicator light 155 on audiometer 140. Alternatively, user 105 visually indicates (e.g., by raising a hand) that a tone has been heard. The audiologist manually records (e.g., on a written chart) the frequency and amplitude of each tone produced and the response of user 105 to those tones. This information is represented by a graph called an audiogram, which represents the threshold of hearing of user 105 for a plurality of audio frequencies.

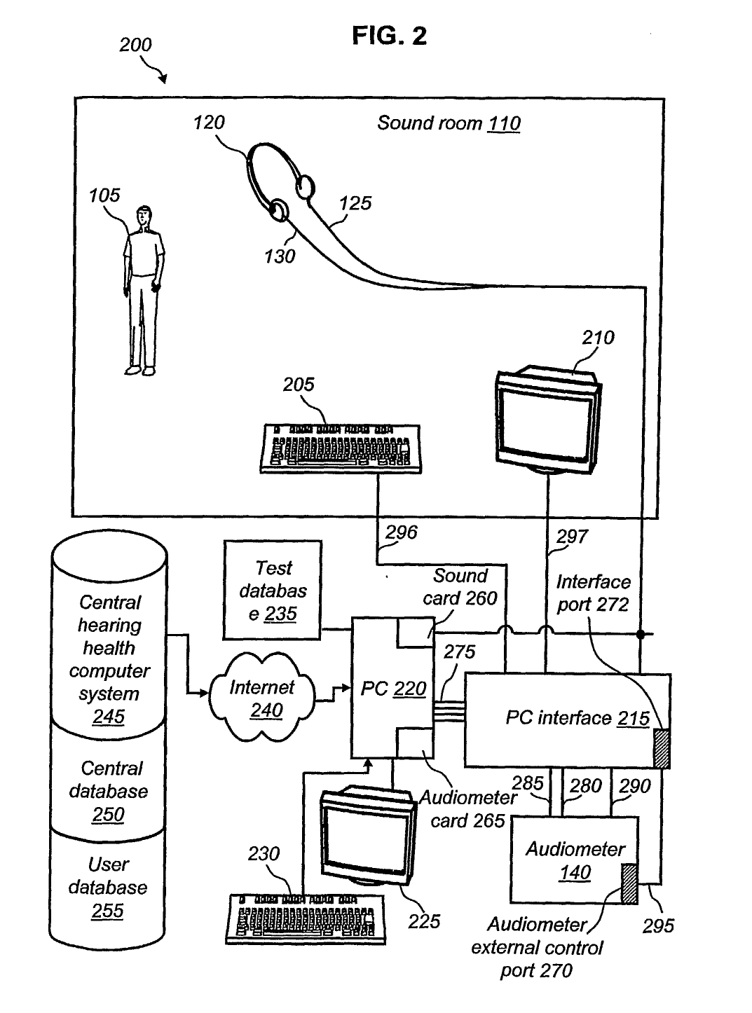

FIG. 2 is a block diagram illustrating a computer-interfaced audiometer system 200 according to the present invention. System 200 incorporates user 105, sound room 110, headphones 120, leads 125 and 130, and audiometer 140 as described in reference to FIG. 1. System 200 further includes a user keyboard 205, a user monitor 210, a PC interface 215, a personal computer (PC) 220, a monitor 225, a keyboard 230, a test database 235, an Internet 240 connection, a central hearing health computer system 245, a central database 250, a user database 255, a digital connection 275, a right signal line 280, a left signal line 285, a switch line 290, a cable 295, a user input 296, and a monitor cable 297.

PC interface 215 provides the interface between audiometer 140, PC 220, headphones 120, keyboard 205, and monitor 210. The connections between PC interface 215 and audiometer 140, PC 220, headphones 120, keyboard 205, and monitor 210 are any conventional means, such as cables, that support digital connection 275, right signal 280, left signal 285, switch line 290, cable 295, user input 296, or monitor cable 297.

Audiometer 140 is electrically connected to PC interface 215 by right signal 280, left signal 285, switch line 290, and optional cable 295. Right signal 280 and left signal 285 provide input of data from audiometer 140 to PC interface 215. Switch line 290 provides output of data from PC interface 215 to audiometer 140. Cable 295 is an optional connection that can be used to electrically connect PC interface 215 and audiometer 140 through an interface port 272 in PC interface 215 and an audiometer external control port 270 in audiometer 140. This optional connection provides automated operation of audiometer 140 by PC 220.

PC 220 is electrically connected to PC interface 215 by digital connection 275 (e.g., USB, RS32, parallel I/O, wireless), which transmits data bi-directionally between PC 220 and PC interface 215.

Headphones 120 are electrically connected to PC interface 215 by leads 125 and 130. Keyboard 205 is electrically connected to PC interface 215 by user input 296. Monitor 210 is electrically connected to PC interface 215 by monitor cable 297.

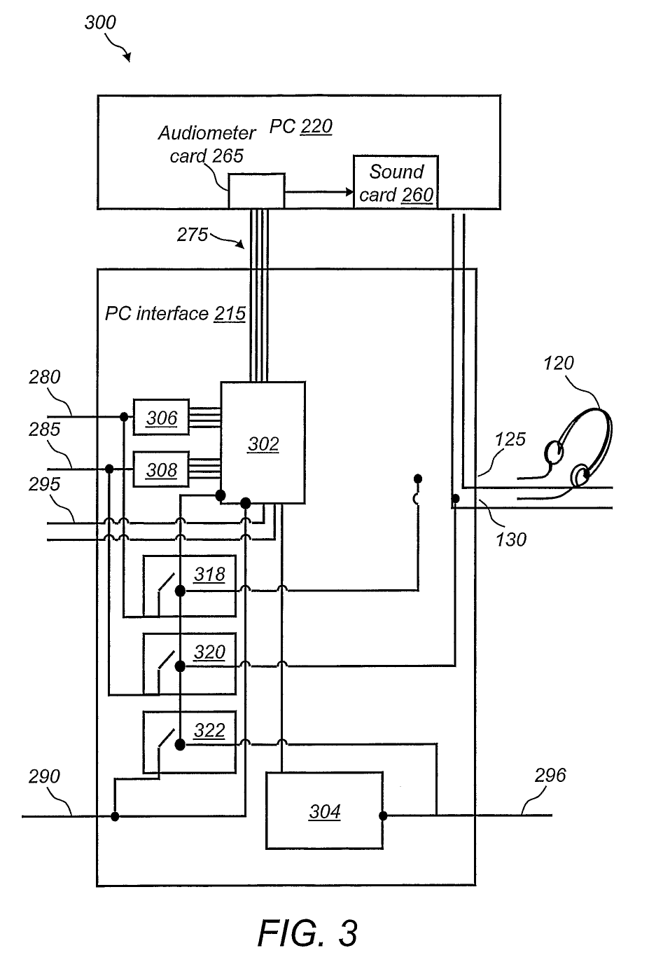

The electrical connections between PC interface 215 and audiometer 140, PC 220, headphones 120, and keyboard 205 are further described in reference to FIG. 3.

Keyboard 205 is a response device that is used by user 105 to indicate that a sound (tone) has been heard. Monitor 210 is an optional component of system 200 that can be used to display questions or information from the audiologist to user 105.

PC 220 is the central input-output processing unit (that includes keyboard 205, monitor 210, keyboard 230, monitor 225, and all PC-related hardware such as disk drives, memory, modems, or connection means, all not shown). Monitor 210, monitor 225, and keyboard 205, keyboard 230 are output and input devices, respectively, for PC 220.

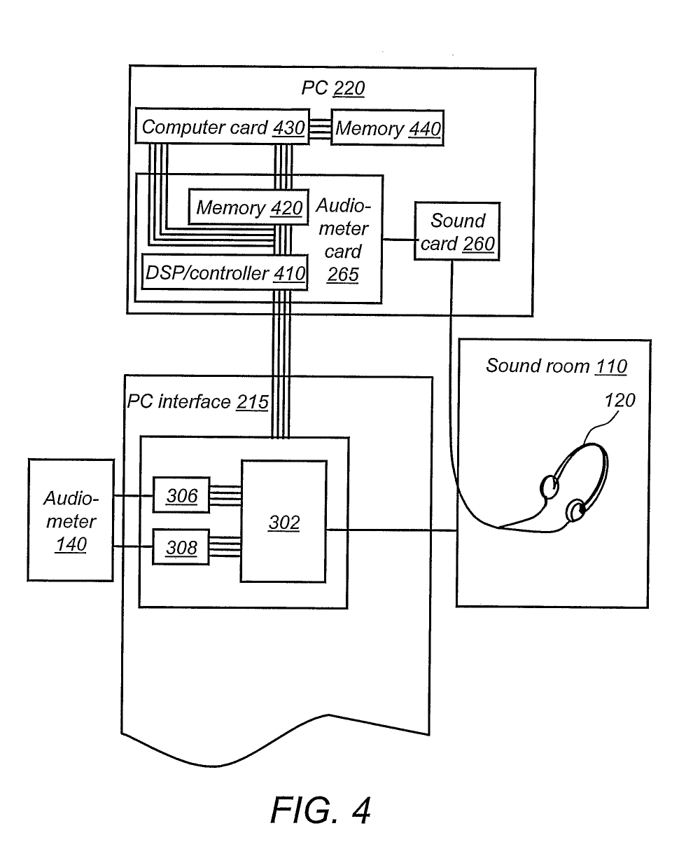

PC 220 further includes an audiometer card 265 and a sound card 260. Audiometer card 265 collects and stores data from audiometer 140 and responses from user 105, as described in reference to FIG. 4. Sound card 260 simulates the sound for a hearing test.

Central hearing health computer system 245 is a remote system that is connected to PC 220 through Internet 240.

Internet 240 is a standard Internet connection, or alternatively is a WAN, LAN, etc. Internet 240 is the communication infrastructure between PC 220 and central hearing health computer system 245. Internet 240 allows central hearing health computer system 245 to remotely administer hearing aid tests, thereby allowing central hearing health computer system 245 the opportunity to reach a large number of individuals.

PC 220 further contains test database 235 to store information such as patient profiles, hearing amplification tables, and patient test results. Test database 235 also stores information such as software programs and information that is downloaded from central hearing health computer system 245.

Central hearing health computer system 245 is a centrally located computer system that is connected to Internet 240, and is capable of performing all normal computer functions, such as reading and writing data to memory (within central hearing health computer system 245), reading and writing data to PC 220, communicating through modem or network connections, and running user test programs. Central hearing health computer system 245 is a central repository of all current audiological programs, audiological data, audiological research, sound “.wav” files, and speech and other sound simulations files. Central hearing health computer system 245 centralizes information such that all connected audiologists around the world can access the current audiological test procedures, new standards, and new algorithms for programming devices such as DSP-based hearing aids.

User database 255 is a memory region of central hearing health computer system 245 that stores user data such as demographics information (age, name, date of birth, etc.), but also includes the user's actual responses to the hearing tests. Central database 250 is another memory region of central hearing health computer system 245, and stores user test programs (not shown).

FIG. 3 is a circuit diagram 300 of PC interface 215 and system 200. PC interface 215 further includes a controller 302, an interface circuit 304, a pair of analog to digital converters (ADC) 306 and 308, and a plurality of switches 318, 320, and 322.

Controller 302 is electrically connected to ADC 306, ADC 308, PC 220, interface circuit 304, switch line 290, and optional cable 295. Controller 302 routes data from audiometer 140 (i.e., right signal line 280, left signal line 285, cable 295) and keyboard 205 or button 135 (i.e., user input line 296) to PC 220. Controller 302 also routes data from PC 220 to audiometer 140 (i.e., cable 295, switch line 290) and headphones 120 (i.e., leads 125 and 130).

Interface circuit 304 normalizes (i.e., between zero and five volts) input voltage from user input line 296 before outputting data to controller 302.

ADC 306 and ADC 308 convert analog voltage transmitted by right signal line 280 and left signal line 285, respectively, to digital data that is output to controller 302.

Switches 318 and 320 redirect input data from right signal line 280 and left signal line 285, respectively, directly to leads 125 and 130, respectively (i.e., switches 318 and 320 are “on”). Switch 322 redirects input data from user input line 296 directly to switch line 290. Switches 318, 320, and 322 can be manually controlled using external buttons or can be programmed through PC 220.

PC interface 215 provides three modes of the invention to audiometer 140. In the first mode of the invention, PC interface 215 provides a direct connection between audiometer 140, headphones 120, and button 135 (i.e., PC interface 215 bypasses PC 220), and audiometer 140 is operated as a conventional audiometer. In the second mode of the invention, PC interface 215 provides an interface between audiometer 140 and PC 220, and an audiologist operates audiometer 140 using PC 220. PC 220 provides data acquisition and retrieval functions from local and centralized sources. In the third mode of the invention, PC interface 215 provides automated control of audiometer 140 by PC 220.

In the first mode of the invention, switches 318, 320, and 322 provide conventional operation of audiometer 140. When switches 318, 320, and 322 are turned on either manually or by PC 220, audiometer 140 is directly connected to headphones 120 and button 135.

In the second mode of the invention, right signal line 280 and left signal line 285 route analog data from audiometer 140 to PC interface 215. ADC 306 and ADC 308 convert analog voltage transmitted over right signal line 280 and left signal line 285, respectively, to digital data that is output to controller 302. Controller 302 routes the digital data received from ADC 306 and ADC 308 to audiometer card 265 in PC 220 via digital connection 275. Audiometer card 265 outputs data to sound card 260. Sound card 260 generates a tone that is transmitted via leads 125 and 130 to headphones 120. Audiometer card 265 also provides data acquisition and retrieval functions that are further described in reference to FIG. 4.

In the third mode of the invention, PC interface 215 provides automated control of audiometer 140 by PC 220. Cable 295 (a set of wires) is an optional connection that is used to electrically connect PC interface 215 and audiometer 140 via interface port 272 and audiometer external control port 270. This optional connection provides automated operation of audiometer 140 (e.g., automatic setting of frequency and amplitude).

FIG. 4 is high-level block diagram of audiometer card 265. Audiometer card 265 further includes a digital signal processor (DSP)/controller 410 and a memory 420. PC 220 further includes a computer card 430 and a memory 440.

DSP/controller 410 is electrically connected to controller 302 and computer card 430. DSP/controller 410 is a real-time processor that enables determination of the frequency and amplitude of output data from controller 302. DSP/controller 410 also provides attenuation and amplification of frequencies from controller 302. Digital frequency and amplitude data is stored in memory 420.

Memory 420 is electrically connected to DSP/controller 410 and computer card 430. Memory 420 provides data storage to DSP/controller 410. Data stored in memory 420 includes frequency and amplitude data from audiometer 140 and user input data from keyboard 205 or button 135.

Memory 440 provides data and program (e.g., software) storage to computer card 430. Programs stored in memory 440 are output to computer card 430 and used to run a program.

Computer card 430 executes programs stored in memory 440. Examples of programs include acquisition and analysis of frequency and amplitude input data from audiometer 140 and acquisition and recording of user input data from keyboard 205 or button 135. Data processing by DSP/controller 410 and programs executed by computer card 430 provide data acquisition and analysis that eliminate the need for manual recording of frequency, amplitude, and user responses required in conventional audiometer testing systems.

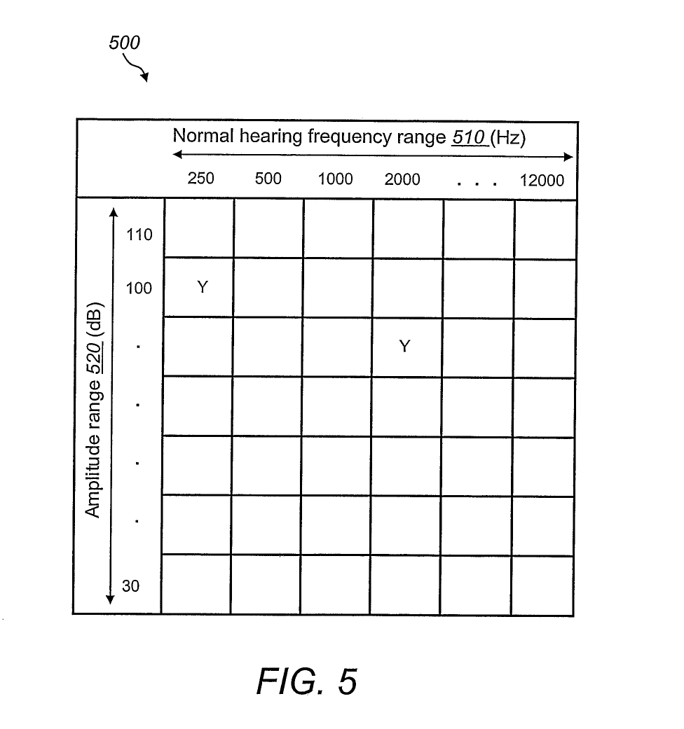

FIG. 5 illustrates a table 500 typically used to document a hearing test. Table 500 includes a normal hearing frequency range 510 and an amplitude range 520.

Humans hear at frequencies ranging from 15 to 20,000 Hz. Normal hearing frequency range 510 shows a smaller range from 250 to 12,000 Hz. During a hearing test as described in reference to FIG. 1 or FIG. 2, an audiologist may choose to test sounds of different frequency ranges across a series of amplitudes. Amplitude range 520 shows a typical range of 30 to 110 decibels (dB). A positive response (i.e., a tone was heard) by user 105 is recorded as a “yes” or “Y” at the appropriate intersection of frequency and amplitude. PC 220, when interfaced to audiometer 140, automatically generates table 500.

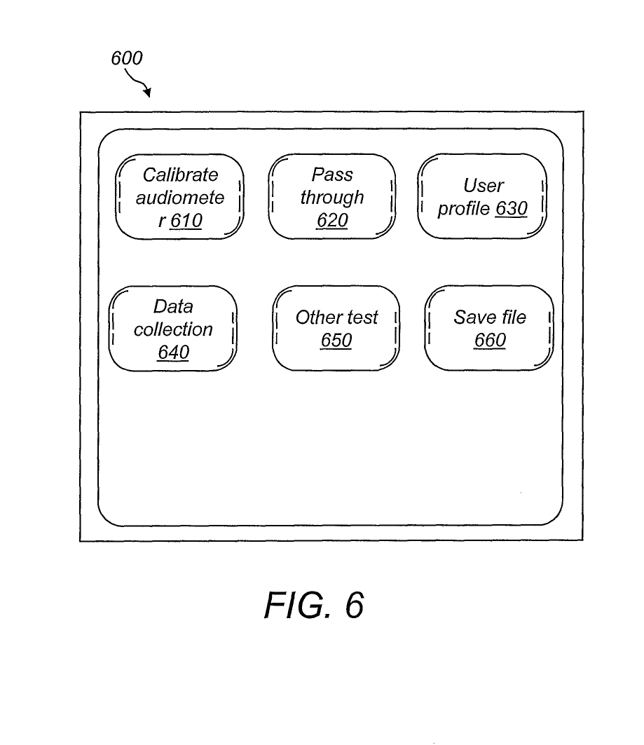

FIG. 6 illustrates a graphical user interface (GUI) 600 associated with the testing system. GUI 600 is displayed on monitor 225 and enables the audiologist to initiate a program with the touch of a finger or stylus or the click of a mouse. GUI 600 includes a plurality of command buttons, including a calibrate audiometer 610, a pass through 620, a user profile 630, a data collection 640, an other test 650, and a save file 660.

Calibrate audiometer 610 initiates a program stored in PC 220 that contains a set of instructions for calibrating audiometer 140. For example, the set of instructions can direct the audiologist to set audiometer 140 to frequency 1000 Hz and amplitude 50 dB and hit “yes” when done. When the audiologist enters “yes”, audiometer 140 sends the appropriate tone, and data representative of the tone is then recorded by PC 220. Various frequencies and amplitudes are entered until the calibration is complete.

Pass through 620 initiates a program stored in PC 220 that turns on switches 318, 320, and 322 to bypass the interface with PC 220, sending tones from audiometer 140 directly to headphones 120 and sending responses from user 105 directly to audiometer 140.

User profile 630 initiates a program stored in PC 220 that allows the audiologist to enter pertinent information about user 105 such as social security number, age, and address.

Data collection 640 initiates a program stored in PC 220 that directs data collection and analysis, and generates table 500.

Other test 650 initiates a program stored in PC 220 that contains a list of other relevant questions such as standard hearing test questions regarding environmental issues of hearing. For example, a standard question may address background noise impact to the ear.

Save file 660 initiates a program stored in PC 220 that contains a set of instructions directing the audiologist to name a file and designate a specified location to store the file.

In operation, an audiologist determines whether to operate computer-interfaced audiometer system 200 as a conventional audiometer or as a computer-interfaced audiometer. To operate computer-interfaced audiometer system 200 as a conventional audiometer, the audiologist turns on switches 318, 320, and 322 either manually (e.g., via external buttons) or by a program stored in PC 220. Computer-interfaced audiometer system 200 operates as a conventional audiometer as described in reference to FIG. 1.

To operate computer-interfaced audiometer system 200, the audiologist links to central hearing health computer system 245 through PC 220 and Internet 240 to upload any current information from central database 250 and user database 255, which is then loaded and stored on test database 235. The audiologist calibrates audiometer 140 and conducts the hearing test by operating GUI 600 controls displayed on monitor 225. With headphones 120 on user 105, the audiologist administers a hearing test using audiometer 140 to generate tones at various amplitudes and frequencies, which are transmitted through PC 220 to headphones 120 via leads 125 and 130. The transmitted amplitudes and frequencies are detected and recorded in the memory of PC 220. Using keyboard 205, user 105 responds or fails to respond to each tone transmitted to headphones 120. PC 220 records the responses of user 105 and stores the responses appropriately in a table (e.g., table 500) listing the various tone amplitudes and frequencies utilized during the hearing health test.

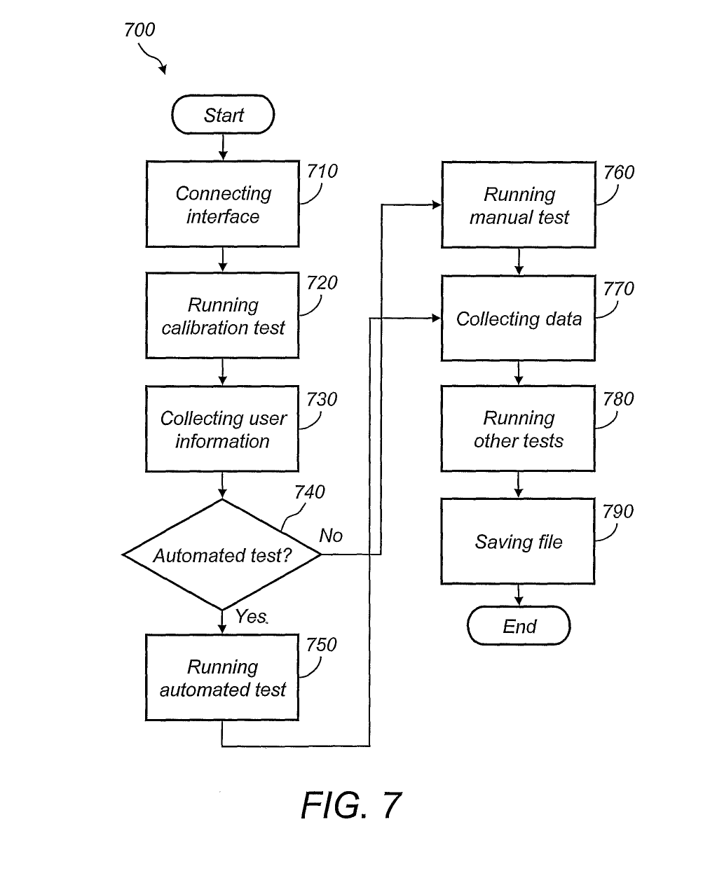

FIG. 7 illustrates a method 700 of using computer-interfaced audiometer system 200 to conduct a standard hearing test. Method 700 includes the steps of:

Step 710: Connecting Interface

In this step, the audiologist electrically connects PC interface 215 to audiometer 140, PC 220, headphones 120, keyboard 205, and monitor 210. Audiometer 140 is connected to PC interface 215 via right signal line 280, left signal line 285, switch line 290, or optional cable 295. PC 220 is connected to PC interface 215 via digital connection 275. Headphones 120 are connected to PC interface 215 via leads 125 and 130. Keyboard 205 is connected to PC interface 215 via user input 296. Monitor 210 is connected to PC interface 215 via monitor cable 297.

Step 720: Running Calibration Test

In this step, the audiologist calibrates audiometer 140 by selecting calibrate audiometer 610 on GUI 600 and following the set of instructions. For example, the set of instructions can direct the audiologist to set audiometer 140 to frequency 1000 Hz and amplitude 50 dB and hit “yes” when done. When the audiologist enters “yes”, audiometer 140 sends the appropriate tone and data representative of the tone is then recorded by PC 220. Various frequencies and amplitudes are entered until the calibration is complete. Calibration allows PC 220 to normalize the incoming signal from audiometer 140 to its own internal DSP calculation of what that frequency is.

Step 730: Collecting User Information

In this step, the audiologist collects pertinent information about user 105 by selecting user profile 630 on GUI 600 and entering the requested data. Additional information about user 105 can be uploaded from central database 250 and user database 255. This information is then loaded and stored on test database 235.

Step 740: Automated Test?

In this decision step, the audiologist determines whether the hearing test will be an automated hearing test (i.e., audiometer 140 can support automated operation). If yes, method 700 proceeds to step 750. If no, method 700 proceeds to step 760.

Step 750: Running Automated Test

In this step, the audiologist ensures that cable 295 is connected to audiometer external control port 270 and interface port 272. With headphones 120 on user 105, the audiologist initiates hearing test software stored on PC 220 that automatically tests the frequency, amplitude, and speech ranges required for a standard hearing test. Method 700 proceeds to step 770.

Step 760: Running Manual Test

In this step, the audiologist, using PC 220, conducts the hearing test stored on PC 220. The audiologist conducts the hearing test by manually adjusting frequency adjust 145 and amplitude adjust 150 on audiometer 140 to the standard range of frequencies and amplitudes required for a standard hearing test (see table 500 of FIG. 5).

Step 770: Collecting Data

In this step, the tone data sent from audiometer 140 and the response of user 105 either via keyboard 205 or button 135 are processed and recorded by PC 220.

Step 780: Running Other Tests

In this step, the audiologist collects other pertinent information for the hearing test by selecting other test 650 on GUI 600, which initiates a program stored in PC 220 that contains a list of other relevant questions to the hearing test such as standard hearing test questions regarding environmental issues of hearing. For example, a standard question may address background noise impact to the ear.

Step 790: Saving File

In this step, the audiologist selects save file 660 to initiate a program stored in PC 220 that contains a set of instructions directing the audiologist to name a file and designate a specified location to store the file. Method 700 ends.

Although preferred embodiments of the present invention have been described and illustrated, it will be apparent to those skilled in the art that various modifications may be made without departing from the principles of the invention.

What is claimed is:

1. A computer-interfaced audiometer system for connecting a conventional audiometer to a computer system, comprising
a computer directed having a program for automatically record hearing test data to provide extended hearing testing capabilities, and
an interface with other computer systems and a central database so as to ensure rapid and accurate hearing health assessments and testing.

2. The audiometer system of claim 1, wherein the audiometer generates analog right and left tone signals at respective right and left signal outputs, the interface comprising:
an interface for coupling the right and left signal outputs, wherein the interface includes an analog to digital converter (“ADC”) for converting analog tone signals to digital tone data.

3. The audiometer system of claim 2, further comprising
a controller including a processor and a memory, wherein the controller is coupled to the interface, a digital signal processor (“DSP”), a sound card, a tone output and an operator input.

4. The audiometer system of claim 3, wherein the processor is selectively controllable to operate the interface in a legacy mode and a processor control mode.

5. The audiometer system of claim 4, wherein in the legacy mode the processor routes the analog tone signals received at the interface to the tone output.

6. The audiometer system of claim 5, wherein in the processor control mode the processor transmits the digital tone data to the DSP.

7. The audiometer system of claim 6, wherein the DSP based on the digital tone data generates frequency and amplitude data corresponding to the analog tone signals represented by the digital tone data, stores the frequency and amplitude data in the memory and transmits the digital tone data to the sound card.

8. The audiometer system of claim 2, wherein the controller is coupled to an input control port of the audiometer and the processor is selectively controllable to operate the interface in an automated processor control mode.

9. The audiometer system of claim 8, wherein in the automated processor control mode the processor and the DSP operate as in the processor control mode and the processor further uses control data transmitted from the operator input to control generation of analog tone signals at the audiometer.

10. The audiometer system of claim 2, wherein the DSP in the processor control mode modifies the digital tone data with respect to at least one of amplitude and frequency characteristics of a corresponding analog tone signal.

11. The audiometer system of claim 2, wherein the interface includes a network communications interface and the DSP generates sound data signals for transmission to the sound card based on hearing testing data received at the network interface.

12. The audiometer system of claim 2, wherein a user input is coupled to the controller and the processor stores in the memory data transmitted from the user input.

13. A method for using a computer-interfaced audiometer system for connecting a conventional audiometer to a computer system, comprising the steps of
providing a computer having a program for automatically recording hearing test data for providing extended hearing testing capabilities, and
providing an interface with other computer systems and a central database so as to ensure rapid and accurate hearing health assessments and testing.

14. A method for using an interface for an audiometer generating analog right and left tone signals at respective right and left signal outputs, and a computer system for data analysis and audiological testing, the method comprising:
providing an interface for coupling the right and left signal outputs, wherein the interface includes an analog to digital converter (“ADC”) for converting analog tone signals to digital tone data;
providing a controller including a processor and a memory, wherein the controller is coupled to the interface, a digital signal processor (“DSP”), a sound card, a tone output and an operator input;
wherein the processor is selectively controllable to operate the interface in a legacy mode and a processor control mode,
wherein in the legacy mode the processor routes the analog tone signals received at the interface to the tone output, and
wherein in the processor control mode the processor transmits the digital tone data to the DSP, wherein the DSP based on the digital tone data generates frequency and amplitude data corresponding to the analog tone signals represented by the digital tone data, stores the frequency and amplitude data in the memory and transmits the digital tone data to the sound card.

15. The method of claim 14, wherein the controller is coupled to an input control port of the audiometer and the processor is selectively controllable to operate the interface in an automated processor control mode, wherein in the automated processor control mode the processor and the DSP operate as in the processor control mode and the processor further uses control data transmitted from the operator input to control generation of analog tone signals at the audiometer.

16. The method of claim 15, wherein the DSP in the processor control mode modifies the digital tone data with respect to at least one of amplitude and frequency characteristics of a corresponding analog tone signal.

17. The method of claim 16, wherein the interface includes a network communications interface and the DSP generates sound data signals for transmission to the sound card based on hearing testing data received at the network interface.

18. The method of claim 17, wherein a user input is coupled to the controller and the processor stores in the memory data transmitted from the user input.
